(19) United States
(12) Patent Application Publication
(10) Pub. No.: US 2005/0096698 A1
(43) Pub. Date: May 5, 2005

(54) Suture needles and methods of use

(76) Inventor: Andrew B. Lederman, Tucker, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/695,886

(22) Filed: Oct. 29, 2003

(51) Int. Cl. A61B 17/06

(52) U.S. Cl. 606/222

(57) ABSTRACT

A suture needle is disclosed that includes a shaft, the shaft being marked at least partially with a visual indicator; and a puncture tip at one end of the shaft. The shaft of the needle includes a front surface and a rear surface. The front and/or rear surface of the needle includes the visual indicator. The visual indicator may be a color, but if both the front and rear surfaces include a color, then the colors of front and rear surfaces are different. Also disclosed are methods of using a suture needles, one such method including: inserting the suture needle into a mammal; viewing the suture needle; and immediately ascertaining the exact orientation of the suture needle with no manipulation of the suture needle.

### BACKGROUND

1. Field of the Invention

This disclosure is generally related to surgical needles and, more particularly, is related to colored suture needles and methods for using colored needles.

2. Description of Related Art

One of the prime problems encountered in many surgical procedures is the difficulty which the surgeon and surgical assistants have in clearly seeing the surgical devices being employed. This problem is especially acute in surgical procedures such as are employed in operations of the eye, the inner ear, etc. and in videoscopic or video-assisted procedures such as laparoscopic surgery. The visibility problem is also especially acute during the suturing phases of such procedures.

In various forms of surgery, including endoscopic or video-assisted surgery, a surgical needle is used, with a suture material or thread secured to the end opposite the puncture tip. Such surgical needles are generally known and, in most cases, consist of a corrosion-resistant metal, preferably of chrome-nickel steel. With such needles, whose whole length is bare or has not been surface-treated, precise establishment of the puncture point and three-dimensional orientation of the needle is frequently problematic during an operation. In addition, when pulling out the needle after the tissue has been pierced, determination of how much further the needle must still be pulled until its rear section with the thread attachment has also emerged from the tissue is, in many cases, possible only with difficulty.

In many cases, the surgical needles have had a bright or shiny surface, a result of polishing, which for the most part was required in order to obtain desired sharpness and cutting characteristics or penetration characteristics with the needle. A drawback to these shiny surfaces is the difficulty they present in being observed by the surgeon during a surgical procedure. Cardiovascular surgeons and micro-surgeons have found it quite difficult to use needles which reflect light in surgery because of the reduced visibility of such needles. With the advent of micro-surgery and video-assisted surgery, there is a similar problem in lack of visibility within the surgical site of these highly reflective needles.

Methods of blackening the reflective surfaces of metal materials have been known for sometime and have been attempted with surgical needles. These blackening processes suffer from one or more disadvantages. Often these blackening processes form a coating on the needle that may flake off during use. In some instances the processes form a non-uniform non-reflective surface which causes visibility problems as well as interfering with the sharpness characteristics of the needle. Most notably, even with a darkened needle, as with a shiny needle, it is nearly impossible for a surgeon to tell the direction the needle is pointing merely by looking at the needle, particularly when the needle is viewed on a monitor as in videoscopic procedures.

For example, FIGS. 1 and 2 show the same suture needle 100 commonly used in surgical procedures. In both FIGS. 1 and 2, the needle 100 includes a curved cylindrical shaft 110 and a puncture tip 120. The shaft 110 includes a front surface 130 and a rear surface 140. For purposes of illustration, a suture thread 150 is attached to the needle 100. In FIG. 1, the needle 100 is pointed with the puncture tip 120 at an orientation of approximately 60° (degree) angle of rotation away from a viewer. In FIG. 2, the needle 100 is pointed with the puncture tip at an orientation of approximately 120° (degree) angle of rotation away from the same viewer perspective.

As can be seen, the needle 100 looks identical to a viewer in both FIGS. 1 and 2, even though the puncture tip 120 of FIG. 1 is actually much closer to the viewer than the puncture tip 120 in FIG. 2. This is especially problematic for endoscopic, laparoscopic or other video-assisted surgical procedures where the surgeon manipulates the needles via a needle holder and views the needle through a video camera. Endoscopic or video-assisted surgery, including laparoscopic surgery, is a procedure whereby instead of opening the abdomen or other body cavity by incision to provide open access and direct vision of the surgical site, the surgery is performed by inserting the surgical instruments and a video camera into the body through small incisions or via a natural orifice such as the mouth or anus. The small size of the incisions that accommodate the instruments and camera results in less tissue damage, less pain, and faster healing than for traditional open surgery, but limit the surgeon's vision of the surgical site to what can be seen via the video camera and monitor.

For most endoscopic procedures today a video camera is used to televise the surgical site, the enlarged video image appearing upon an external screen or monitor and guiding the surgeon or surgical team in manipulating the instruments through the incisions or orifice. The use of a standard surgical video system removes the ability of the surgeon to visualize the surgical site with three-dimensional depth perception. This limits the surgeon's ability to immediately determine the three-dimensional orientation of the needle and to determine in which direction the puncture tip 120 is pointing. Perception of needle orientation in three dimensions is further limited by the degraded tactile sensation of using long, mechanical or robotically controlled instruments inserted into the body. Although the surgical needles and methods of using them of the prior art are adequate for their intended purpose, there is a need in the art for improved suture needles. In particular, there is a need to provide improved, novel suture needles, along with improved uses for the suture needles that provide ready perception to a viewer of the direction or orientation of the needle, particularly if the needle is being viewed through a video camera.

### SUMMARY

Embodiments of this disclosure provide surgical devices, namely suture needles, and methods for using the suture needles. Briefly described, one embodiment among others, of such a suture needle includes a shaft, the shaft being marked at least partially with a visual indicator, and a puncture tip at one end of the shaft.

One embodiment of a method for using a needle includes: inserting the suture needle into a mammal, viewing the suture needle, and immediately ascertaining the exact orientation of the suture needle with no manipulation of the suture needle.

### BRIEF DESCRIPTION OF THE DRAWINGS

The patent or application file contains at least one drawing executed in color. Copies of this patent or patent application publication with color drawings will be provided by the Office upon request and payment of the necessary fee.

Many aspects of this disclosure can be better understood with reference to the following drawings. The components in the drawings are not necessarily to scale. Moreover, like reference numerals designate corresponding parts throughout the several views.

FIG. 1 is a color illustration of a prior art suture needle, with the puncture tip viewed at a 60° angle of rotation away from a viewer.

FIG. 2 is a color illustration of the prior art suture needle shown in FIG. 1, with the puncture tip viewed at a 120° angle of rotation away from a viewer.

FIG. 3 is a color illustration of an embodiment of the disclosed suture needle, with the rear surface viewed at a 180° angle of rotation away from a viewer.

FIG. 4 is a color illustration of the suture needle shown in FIG. 3, with the rear surface viewed at a 0° angle of rotation away from a viewer.

FIG. 5 is a side view illustrating the suture needle shown in FIG. 3.

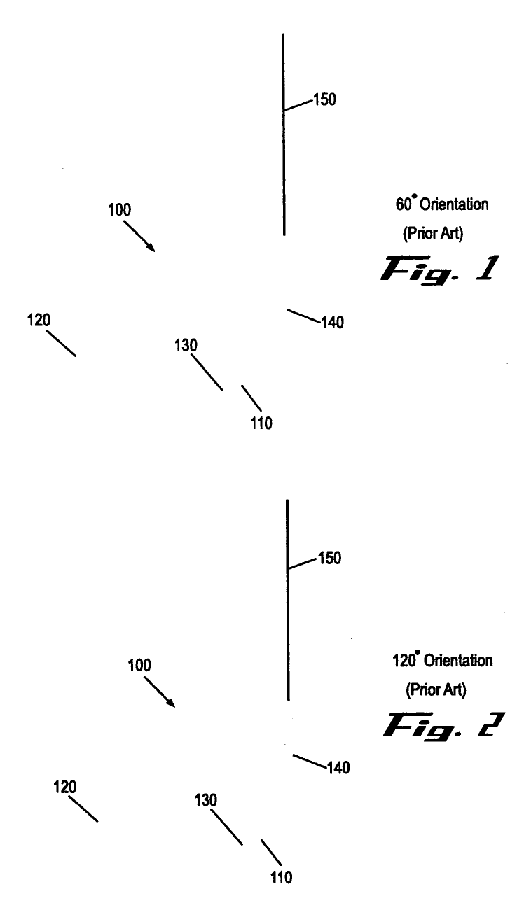

### DETAILED DESCRIPTION

As identified in the foregoing, suture needles and methods for using them are not always sufficient to allow proper visualization of the direction the needle is pointing. In attempting to solve this problem, other suture needles may be harmful or just as difficult to use, or a surgeon may need to manipulate the needle in order to orient it. Therefore, needed are suture needles that allow a surgeon to better visualize or perceive the direction of the needle during the surgical procedure.

Disclosed herein are suture needles and methods of using the suture needles. The disclosed suture needles provide ready cues for the three-dimensional orientation of the needle when placed in the body of a mammal, thus enabling a surgeon to immediately manipulate the suture needle through the tissue of the mammal without having to first determine the orientation of the suture needle.

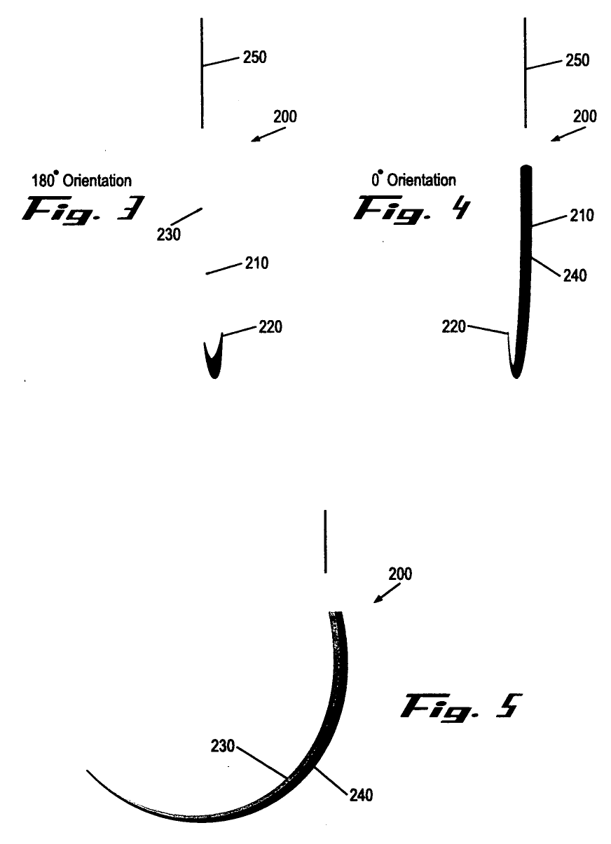

Referring now to the figures, FIG. 3 illustrates an exemplary embodiment of the suture needle 200. The representative suture needle 200 includes a curved cylindrical shaft 210 and a puncture tip 220 at one end of the curve of the shaft 210. The shaft includes a front surface 230. For purposes of illustration, although not a necessary feature of the suture needle 200, a suture thread 250 is depicted in FIG. 3 attached to the shaft 210 of the suture needle 200. FIG. 4 shows a rear view of the suture needle 200, illustrating that the shaft 210 has a rear surface 240. Due to the presence of a visual indicator on the front surface 230, a viewer of the needle 200 can easily determine if the rear surface 240 of the suture needle 200 is rotated 180° away from the viewer as in FIG. 3, where the puncture tip 220 is disposed between the viewer and the shaft 210. Alternatively, or in addition to the visual indicator on the front surface 230, the needle 200 may have a visual indicator on the rear surface 240 that indicates to a viewer that the rear surface 240 of the suture needle 200 is rotated 0° away from the viewer as in FIG. 4, the rear surface 240 being disposed between the viewer and the puncture tip 220.

When viewed from the side, as illustrated in FIG. 5, it can be seen that the front surface 230 of the shaft 210 is proximal the puncture tip 220, meaning the front surface 230 is inside the curve of the shaft 210. The rear surface 240 of the shaft 210 is distal the puncture tip 220, meaning the rear surface 240 is outside the curve of the shaft 210. While a curved suture needle 200 is depicted in FIGS. 3-5, other needles may be marked with a visual indicator, and will have different definitions of the front and rear surfaces.

Suture needles 200 may be fabricated from metals such as stainless steel alloys that have desired characteristics with respect to biocompatibility, strength, and the ability to take a sharp end and/or point when ground and polished. Generally, the needle can be fabricated from a material such as series 300 stainless steel alloy, series 400 stainless steel alloy, or nonferrous alloy, e.g., MP35N alloy. Further, as noted above, while FIGS. 3-5 depict a suture needle with a curved shape, other needles may advantageously be marked with a visual indicator, such as for example, straight, ski, cutting, and/or tapered needles. Advantages of having the two front and rear surfaces 230 and 240 of the shaft 210 being readily separately identifiable will be described in more detail below.

As can be clearly seen in FIGS. 3 and 4, the particular surface of the suture needle 200 that is facing a viewer is readily discernible to the viewer, due to the particular visual indicator that the viewer sees, whether the surface be the front surface 230 or the rear surface 240. While the suture needle 200 is shown to have two different visual indicators on both the front surface 230 and the rear surface 240, an alternative suture needle 200 may only have one visual indicator, or a plurality of visual indicators. For example, in the situation where the suture needle 200 has a visual indicator only on one surface, the viewer is nonetheless able to tell the orientation of the suture needle 200, depending on whether the visual indicator is viewed or not. While FIGS. 3-5 depict the suture needle 200 having visual indicators covering the entire front and rear surfaces of the shaft 210, only a portion of the front surface 230 and/or rear surface 240 may be marked with a visual indicator.

While FIGS. 3-5 depict the suture needle 200 having visual indicators that are either blue or yellow, each visual indicator may also be any color or any combination of colors, or any indicia that would serve to identify or distinguish one surface of the needle from another. For example, the visual indication can also be accomplished by stripes, plaid, checkerboard, speckled, or various patterns, symbols, letters, numbers, or any design or representation that would indicate to a viewer that a particular surface of the suture needle 200 is either viewed or not viewed. By understanding the visual indicator, and upon determining whether it is viewed or not viewed, cues are given to a viewer as to the orientation of the suture needle 200 without a need for manipulation of the suture needle 200. Additionally or alternatively to the above representations, the visual indicator may be a surface treatment, for example, a darkened, raised, matte, dulled, non-reflective, brushed metal, photoluminescent, or other type of surface that would reflect light differently than another surface of the suture needle 200.

The visual indicator can be applied to the needle 200 by applying, for example, color to one or both sides of the suture needle 200. For example, color or any other type of visual indicator can be impregnated on or within the metal used to form the suture needle 200, or it may be applied after the suture needle 200 is fabricated. If applied after fabrication of the suture needle 200, the visual indicator may be, for example, coated, sprayed, brushed, rolled, heat transferred, laminated, painted, etched, lacquered, adhered, chemically, or electrolytically applied or otherwise affixed to all or a portion of at least one surface of the shaft 210 of the suture needle 200. By way of specific example, color or a matte-finish can be applied chemically by either pickling or etching. The pickling may be accomplished by dipping or matte-dipping the needle. Color or a special finish may also be applied as a visual indicator electrolytically through appropriate anodic or cathodic treatment, with polarity inversion or through alternating current.

The suture needle 200 described above is particularly useful in procedures involving endoscopic or videoscopic surgery. For example, arthroscopic, laproscopic, and thoracoscopic surgeries, as well as some open surgeries, employ a monitor or other viewing device that decreases a viewer's three-dimensional perception of the orientation of the suture needle. In situations in which the surgeon uses the suture needle 200 that includes a visual indicator on a front surface 230 and/a rear surface 240, the surgeon is able, even when looking at the video monitor, to immediately assess the exact orientation of the puncture tip 220. Simplification in determining the orientation of the suture tip 220 helps avoid the situation where the surgeon may have to take the time to manually determine, rather than visually, the orientation of the suture needle 200 and/or puncture tip 220. Because the surgeon has visual cues for the orientation of the suture needle 200, rather than through manipulating the suture needle 200, the possibility of accidentally piercing tissue at an undesired location is also reduced. In addition, those learning to suture for the first time, or learning new techniques for suturing, may also benefit from the disclosed needles 200 and methods for using them.

An exemplar method of using the suture needle 200 includes inserting the suture needle 200 into a mammal or other tissue to be sewn, viewing the suture needle 200, and immediately ascertaining the exact orientation of the suture needle 200 with no manipulation of the suture needle 200. Viewing the suture needle 200 may include viewing an indicator on the suture needle 200, where the indicator is present on the front surface 230 of the suture needle 200, proximal a puncture tip 220 and/or is present on a rear surface 240 of the suture needle 200, distal a puncture tip 220. Immediately ascertaining the exact orientation of the suture needle may include determining the angle of rotation away from a viewer or surgeon of the puncture tip 220 on the suture needle 200.

In addition to aiding in determining the orientation of the suture needle 200, the suture needle 200 may be marked with a visual indicator that keeps track of various needles in a procedure. Different needles may be marked with different visual indicators that would indicate to the user which task or function that particular suture needle 200 will be used to perform.

It should be emphasized that the above-described embodiments of the needle and embodiments of method for using the needle are merely possible examples of implementations. Many variations and modifications may be made to the above-described embodiment(s). All such modifications and variations are intended to be included herein within the scope of this disclosure and protected by the following claims.

1. A suture needle comprising:
a shaft, the shaft being marked at least partially with a visual indicator; and
a puncture tip at one end of the curve of the shaft.

2. The suture needle of claim 1, wherein the shaft is comprised of a front surface that is proximal the puncture tip, the front surface including a visual indicator.

3. The suture needle of claim 2, wherein the visual indicator is a color.

4. The suture needle of claim 1, wherein the shaft is comprised of a rear surface that is distal the puncture tip, the rear surface including a visual indicator.

5. The suture needle of claim 4, wherein the visual indicator is a color.

6. The suture needle of claim 1, wherein the shaft is comprised of:
a front surface that is proximal the puncture tip, the front surface including a first visual indicator; and
a rear surface that is distal the puncture tip, the rear surface including a second visual indicator;
wherein the first visual indicator is visually distinct from the second visual indicator.

7. The suture needle of claim 6, wherein the first visual indicator is a first color and the second visual indicator is a second color.

8. The suture needle of claim 7, wherein approximately the entire front surface is comprised of the first color and approximately the entire rear surface is comprised of a second color.

9. The suture needle of claim 1, wherein the suture needle aids in visual perception of the angle of orientation of the needle.

10. The suture needle of claim 1, wherein the suture needle is chosen from: a needle with a curved cylindrical shaft, a straight needle, a ski needle, a cutting needle, and a tapered needle.

11. A suture needle comprising:
a puncture tip; and
a shaft extending from the puncture tip, the shaft including means for distinguishing between a front surface of the shaft and a rear surface of the shaft.

12. The suture needle of claim 11, wherein the shaft comprises a curved cylindrical shaft extending from the puncture tip, and
wherein the means for distinguishing between a front surface that is proximal the puncture tip and a rear surface that is distal the puncture tip.

13. The suture needle of claim 11, wherein the means for distinguishing between the front surface and the rear surface is a color.

14. The suture needle of claim 11, wherein the suture needle is chosen from: a needle with a curved cylindrical shaft, a straight needle, a ski needle, a cutting needle, and a tapered needle.

15. A method of using a suture needle comprising:
inserting the suture needle into a mammal;
viewing the suture needle; and
immediately ascertaining the exact orientation of the suture needle with no manipulation of the suture needle.

16. The method of claim 15, wherein the step of viewing the suture needle comprises viewing an indicator on the suture needle, where the indicator is only present on the front surface of the suture needle, proximal a puncture tip.

17. The method of claim 15, wherein the step of viewing the suture needle comprises viewing an indicator on the suture needle, where the indicator is only present on the rear surface of the suture needle, distal a puncture tip.

18. The method of claim 15, wherein the step of viewing the suture needle comprises viewing the needle on a video monitor connected to an endoscope, the endoscope being located in the mammal.

19. The method of claim 15, wherein the step of immediately ascertaining the exact orientation of the suture needle comprises determining the angle of rotation of a puncture tip on the suture needle away from a viewer.

20. A method of making a suture needle, comprising the steps of:
providing a suture needle; and
marking at least a portion of the suture needle with a visual indicator.

21. The method of claim 20, wherein the step of marking a portion of the suture needle comprises marking a surface of a shaft of the needle chosen from the front surface and the rear surface.
